(19) United States
(12) Patent Application Publication
(10) Pub. No.: US 2008/0281096 A1
(43) Pub. Date: Nov. 13, 2008

(54) Resolution Process For Preparing (+)-2S,3S)-2-(3-Chlorophenyl)-3,3,3-Trimethyl-2-Morpholinol

(76) Inventors: Michael Anthony Harris, Kent (GB); Alan Negus, Kent (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 10/577,412

(22) PCT Filed: Oct. 25, 2004
(86) PCT No.: PCT/EP04/12095

§ 371 Date: Apr. 26, 2006

(30) Foreign Application Priority Data
Oct. 27, 2003 (GB) 0325051.1

(51) Int. Cl. C07D 265/32

(52) U.S. Cl. 544/173

(57) ABSTRACT

Disclosed is a method for preparing (+)-(2S,3S)-2-(3-chlorophenyl)-3,5,5-trimethyl-2-morpholinol and pharmaceutically acceptable salts such as the (+)-(2S,3S)-2-(3-chlorophenyl)-3,5,5-trimethyl-2-morpholinol hydrochloride salt via dynamic kinetic resolution.

### BACKGROUND OF THE INVENTION

1. Field of the Invention

The present invention relates to a process for making (+)-(2S,3S)-2-(3-chlorophenyl)-3,5,5-trimethyl-2-morpholinol (hereinafter the “(2S,3S) enantiomer”) and pharmaceutically acceptable salts such as the hydrochloride salt of the (2S,3S) enantiomer by dynamic kinetic resolution (DKR).

2. Description of the Prior Art

Bupropion hydrochloride, (±)-1-(3-chlorophenyl)-2-[(1,1-dimethyl-ethyl)-amino]-1-propanone hydrochloride, shown below, is the active ingredient of Wellbutrin® which is marketed in the United States for the treatment of depression. It is also the active ingredient of Zyban® which is marketed in the United States as an aid to smoking cessation. Bupropion is an inhibitor of the neuronal uptake of noradrenaline (NA), and dopamine (DA), and does not inhibit the serotonin transporter or monoamine oxidase. While the mechanism of action of bupropion, as with other antidepressants, is not entirely certain, it is believed that this action is mediated by noradrenergic and/or dopaminergic mechanisms. Early evidence suggested Wellbutrin® to be a selective inhibitor of noradrenaline (NA) at doses that were predictive of antidepressant activity in animal models. (Ascher, J. A., et al., Bupropion: A Review of its Mechanism of Antidepressant Activity. Journal of Clinical Psychiatry, 56: p. 395-401, 1995). A more recent analysis of the research (Stahl, S. M. et al., Primary Care Companion, Journal of Clinical Psychiatry, 6, p. 159-166, 2004) concludes that bupropion does act as a selective dopamine and norepinephrine reuptake inhibitor, with slightly greater functional potency at the dopamine transporter.

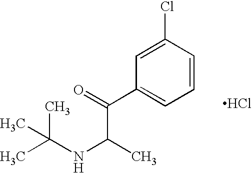

Bupropion is extensively metabolized in man as well as laboratory animals. Urinary and plasma metabolites include biotransformation products formed via hydroxylation of the tert-butyl group and/or reduction of the carbonyl group of bupropion. Four basic metabolites have been identified. They are the erythro- and threo-amino alcohols of bupropion, the erythro-amino diol of bupropion, and a morpholinol metabolite. These metabolites of bupropion are pharmacologically active, but their potency and toxicity relative to bupropion have not been fully characterized. Because the plasma concentrations of the metabolites are higher than those of bupropion, they may be of clinical importance.

The (2S,3S) enantiomer of the morpholinol metabolite (2R*,3R*) racemate has been found to be an active metabolite, and the hydrochloride salt of this enantiomer, as shown below, is a preferred salt.

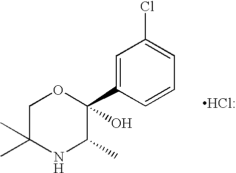

The (2S,3S) enantiomer and pharmaceutically acceptable salts and solvates thereof, and pharmaceutical compositions comprising the same are useful in treating numerous diseases or disorders such as depression, attention deficit hyperactivity disorder (ADHD), obesity, migraine, pain, sexual dysfunction, Parkinson's disease, Alzheimer's disease, or addiction to cocaine, alcohol or nicotine-containing (including tobacco) products. For instance, reference is made to co-pending U.S. application Ser. No. 10/150,287, U.S. Pat. No. 6,342,496 B1, issued to Jerussi et al. on Jan. 29, 2002, U.S. Pat. No. 6,337,328 B1, issued to Fang et al. on Jan. 8, 2002, U.S. Patent Application Publication Nos. 2002/0052340 A1, 2002/0052341 A1, and 2003/0027827 A1 as well as WO 01/62257 A2. The methods of treating these diseases and disorders as described in these references and the references cited therein are herein incorporated by reference.

The references cited in the previous paragraph describe the preparation of either the (2S,3S) or (2R,3R) enantiomer from the (2R*,3R*) racemate. U.S. Pat. No. 6,337,328, U.S. Patent Application Publication Nos. 2002/0052341 A1 and 2003/0027827, and WO 01/62257 A2 refer to a chiral acid resolution method for preparing (2S,3S) enantiomer from the (2R*,3R*) racemate. However, the method described in each of these references differs from the present invention in both procedure and result. These references relate to chemical resolutions of the racemate, whereas the present invention involves DKR which results in the chemical conversion of the (2R,3R) enantiomer to the (2S,3S) enantiomer, so that the yields of the (2S,3S) enantiomer are greater than 50% based on the concentration of the racemic mixture of the (2R,3R) and (2S,3S) enantiomers. In the simple chemical resolution of the racemate, these references must isolate the desired diastereomeric morpholinol from a mixture of diastereomeric salts. The maximum yield of the desired diastereomer can therefore be at most 50% based on the concentration of the mixture of the (2R,3R) and (2S,3S) enantiomers.

In general, most chemical or enzymatic resolutions of a racemic material produce the desired enantiomer or mirror image diastereoisomer in a maximum theoretical yield of 50%. The undesired enantiomer or mirror image diastereoisomer is discarded as waste. In rare cases, a DKR can be employed to give a maximum theoretical yield of 100% of a desired enantiomer via equilibration of the enantiomers during the resolution. However, DKR's are extremely rare for the preparation of single pure diastereoisomers (particularly, for example, compounds containing two chiral centers), since both chiral centers must be capable of equilibration.

### SUMMARY OF THE INVENTION

Accordingly, it is an object of the present invention to provide a novel process for producing a salt of the (2S,3S) enantiomer that is essentially enantiomerically pure from an initial sample comprising the (2R,3R) enantiomer by DKR in a yield of greater than 50% based on the initial sample.

When the present invention is compared with prior methods of isolation, it will be apparent that according to the present invention, there will be a much higher yield of the target compound, the (2S,3S) enantiomer, and the inactive (2R,3R) enantiomer will be present in such low concentrations as to not seriously diminish the pharmaceutical effectiveness of the product.

In one embodiment, the present invention is drawn to a DKR process for preparing a salt of the (2S,3S) enantiomer that comprises:

mixing i) a sample comprising the (2R,3R) enantiomer, ii) at least one solvent having a boiling point of at least 50° C. and iii) 1.1 equivalent or higher of (−)-(R,R)-di-p-toluoyl-L-tartaric acid (hereinafter “L-DTTA”) in any order, heating the mixture to at least 50° C. for at least 1 hour to form crystals comprising the L-DTTA salt of the (2S,3S) enantiomer, and isolating the crystals, wherein the yield of the L-DTTA salt of (2S,3S) enantiomer is greater than 50% based on said sample.

### DETAILED DESCRIPTION OF THE INVENTION

The present invention provides a method for making the (2S,3S) enantiomer, a single diastereoisomer from a two-chiral center racemate. The process is an example of a crystallization-induced asymmetric transformation, also termed a second-order asymmetric transformation, but, importantly with two chiral centers equilibrating. (For one chiral center equilibrating asymmetric transformations see “Crystallization-Induced Asymmetric Transformations” in Jacques, J., Collet, A. and Wilen, S. H., in Enantiomers. Racemates and Resolutions, Krieger Publishing Company, Malabar, Fla., 1991, Chapter 6, pp. 369-377). These processes are also referred to as DKR as disclosed in “Enantioselective Synthesis: The Optimum Solution”, Partridge, J. J. and Bray, B. L. in Process Chemistry in the Pharmaceutical Industry, (Gadamasetti, K. G., Ed.) Marcel Dekker, New York, N.Y., 1999, pp. 314-315.

In one embodiment, the process for preparing a salt of the (2S,3S) enantiomer comprises:

mixing i) a sample comprising the (2R,3R) enantiomer, ii) at least one solvent having a boiling point of at least 50° C. and iii) 1.1 equivalent or higher of L-DTTA in any order, heating the mixture to at least 50° C. for at least 1 hour to form crystals comprising the L-DTTA salt of the (2S,3S) enantiomer, and isolating the crystals, wherein the yield of the L-DTTA salt of (2S,3S) enantiomer is greater than 50% based on said sample.

The solvent for use in the inventive process can be any type, so long as the solvent will preferably dissolve the L-DTTA salt of the (2R,3R) enantiomer over the L-DTTA salt of the (2S,3S) enantiomer. Preferably the solvent has a boiling point of at least 50° C. More preferably, the solvent has a boiling point of 55-110° C. Most preferably, the solvent is at least one selected from the following: alkyl acetate, such as methyl acetate, ethyl acetate (sometimes referred to herein as “EtOAc”), isopropyl acetate, propyl acetate, butyl acetate; dialkyl ketone such as 2,4-dimethyl-3-pentanone, 3-methyl-2-butanone, 2-butanone and 4-methyl-2-pentanone; and a nitrile such as acetonitrile and propionitrile. In an embodiment the solvent is ethyl acetate.

The molar amount of L-DTTA relative to the molar amount of the (2R,3R) enantiomer, (or if the (2S,3S) enantiomer is also present relative to the combined molar amount of the (2R,3R) and (2S,3S) enantiomers) is 1.1 equivalents or higher. Preferably, the amount is 1.2-2.0 equivalents. More preferably, the amount is 1.3-1.5 equivalents.

In an embodiment of the invention, the crystallization of the target compound is promoted by adding a seed crystal of a salt of the (2S,3S) enantiomer to said mixture.

The mixture of the sample comprising the (2R,3R) enantiomer, solvent and L-DTTA is heated to at least 50° C. Preferably, the mixture is heated to reflux. While the mixture is being heated, the following equilibrium reaction between the (2R,3R) and (2S,3S) enantiomers proceeds:

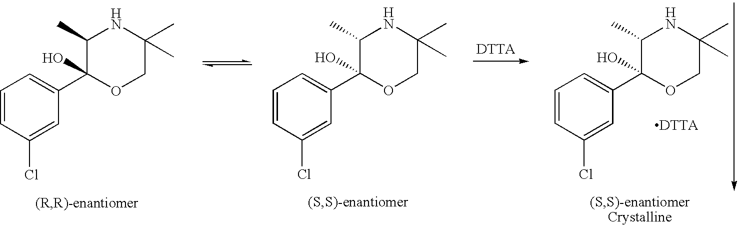

By maintaining the mixture at a temperature of at least 50° C. for a sufficient period of time, the crystallization of the L-DTTA salt of the (2S,3S) enantiomer removes the (2S,3S) enantiomer from the equilibrium thereby driving the equilibrium to the right (as shown above). Preferably, the mixture is heated for at least 1 hour. More preferably the mixture is heated for at least 5 hours. Most preferably, the mixture is heated for 10-16 hours. When a temperature of between 50° C. and about 80° C. is used, heating for 16-24 hours is suitable. Due to the possible equilibrium kinetics, to achieve an effective yield of the desired (2S,3S) enantiomer the temperature at which the mixture is heated and the length of time for which the mixture is heated may be factors which are inversely proportional.

As heating proceeds, the crystals of the L-DTTA salt of the (2S,3S) enantiomer begin to form. These crystals may also contain the undesired (2R,3R) enantiomer (as a salt) based on the type of solvent chosen for the DKR. In other words, the DTTA salt of the undesired (2R,3R) enantiomer may be partially insoluble in the chosen solvent and a portion thereof crystallizes with the DTTA salt of the required (2S,3S) enantiomer. However, the solvents of the present invention will have a much higher preference for dissolving the DTTA salt of the (2R,3R) enantiomer thereby leading to a product having relatively high enantiomeric purity. In the present invention, the enantiomeric purity of the (2S,3S) enantiomer in the crystals of the present invention is at least 80%. Preferably, the enantiomeric purity is at least 92%. More preferably, the enantiomeric purity is at least 96%. Most preferably, the enantiomeric purity is at least 98.5%. As used herein, an “essentially enantiomerically pure” sample, contains the (2S,3S) enantiomer in at least 96%.

Suitably the process of the present invention is performed under conditions in which the water content is kept below 0.5%, or below 0.1%. The person skilled in the art will be aware of steps which can be taken to ensure the water content is kept below such levels. It has been found that under acidic conditions with higher water content (2% and 5%) the racemate degrades (although the chiral purity is unaffected), resulting in contamination of the isolated (2S,3S)-DDTA salt with AMP.DDTA salt(s) of undefined stoichiometry (AMP=2-amino-2-methylpropanol). Degradation is also observed with ethanol and methanol being used as the solvent, and may also be observed to a lesser extent with other solvents.

In an embodiment of the present invention, the process forms the L-DTTA salt of the (2S,3S) enantiomer in a yield of at least 50% based on the initial sample comprising the (2R,3R) enantiomer. Preferably, the yield is at least 60%. Most preferably, the yield is at least 75%.

The isolated yield of the required (2S,3S) enantiomer salt in sufficient purity is important, thus taking into account the degradation aspects referred to above. Hence, achieving a yield of at least 50% of isolated enantiomerically pure (2S,3S) enantiomer salt reflects the practical consequence of an effective dynamic kinetic resolution.

In an embodiment of the present invention, the process further comprises a step of converting the L-DTTA salt of the (2S,3S) enantiomer to another salt. Preferably, said another salt is a pharmaceutically acceptable salt, such as a hydrochloride salt.

The method for preparing the racemate is not particularly limited. The methods described in U.S. Pat. No. 6,342,496 B1, U.S. Pat. No. 6,337,328 B1, U.S. Patent Application Publication Nos. 2002/0052340 A1, 2002/0052341 A1, and 2003/0027827 A1 as well as WO 01/62257 A2 are herein incorporated by reference. A particularly preferred method is now given; however, it should be understood that the specific examples, while indicating preferred embodiments of the invention, are given by way of illustration only, since various changes and modifications within the spirit and scope of the invention will become apparent to those skilled in the art from this detailed description. Suitable methods for converting the L-DTTA salt to another salt will be well-known to the person skilled in the art, with specific methods for conversion to the hydrochloride salt also being disclosed in the above-mentioned patents and applications.

### EXAMPLES

### Synthesis of the Racemate

3′-Chloropropiophenone (25 g, 0.148 mol) was gently stirred and heated to 50° C. until molten. Bromine (23.9 g, 0.149 mol, 1.01 equiv.) was added, keeping the temperature at 50-55° C. The crude bromoketone was gently purged with nitrogen then heated at 75-80° C. for 30 minutes to expel hydrogen bromide.

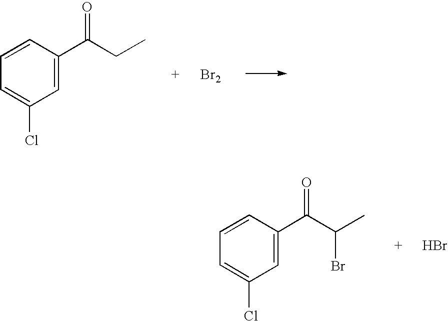

Ensuring the temperature of the bromoketone reaction mixture was below 77° C., ethyl acetate (25 ml) was then added. The solution was heated to reflux (solution temperature approximately 90° C., heating bath at 115° C.), then 95% 2-amino-2-methylpropanol (34.7 g containing 5% water, 0.37 mol, 2.5 equivalents) was added slowly, while maintaining reflux. The mixture was then boiled under reflux for 3.0 hours. The hot mixture was diluted with water (30 ml) then ethyl acetate (35 ml), stirred for 5 minutes, then transferred to a separating funnel, washing with water (45 ml) then ethyl acetate (65 ml). The temperature of the mixture was maintained above 40° C. during workup to minimize the risk of crystallization.

The organic phase was separated then washed with water (75 ml). The solution containing the racemate was concentrated to approximately 64 ml at atmospheric pressure then diluted with fresh ethyl acetate (86 ml). Distillation was continued until a further 86 ml of distillate was collected. The solution was diluted with ethyl acetate (107 ml) then sampled for water determination. If the water content was greater than 0.1% a further 86 ml of ethyl acetate was distilled out. The solution was then diluted to 300 ml (275.8 g) with ethyl acetate.

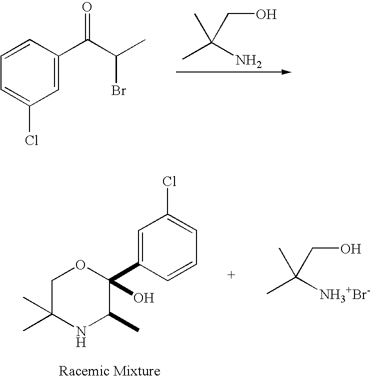

### Synthesis of the Target (2S,3S) Enantiomer

### Example 1

A solution of L-DTTA (74.43 g, 0.192 mol, 1.3 equiv) in ethyl acetate (100 ml) was prepared in a 1000 ml flask and heated to reflux. 45 ml of the solution of racemate in ethyl acetate prepared above was added to the boiling L-DTTA as rapidly as possible. Without delay seed crystals of the L-DTTA salt of the (2S,3S) enantiomer (0.05 g) were added and boiling continued for about 1 hour. The remainder of the solution of racemate in ethyl acetate prepared above was added to the boiling L-DTTA solution over a period of 5 hours, and was rinsed with ethyl acetate (17.8 ml). Reflux was continued for a further 14 hours. The suspension was cooled to ambient temperature. The product was filtered off, washed with ethyl acetate (3×100 ml, some of the wash can be used to wash out the vessel) then dried at 50° C. under vacuum, to give 70.7 g (74% yield based on the 3′-chloropropiophenone starting material) of the L-DTTA salt of the (2S,3S) enantiomer as white crystals.

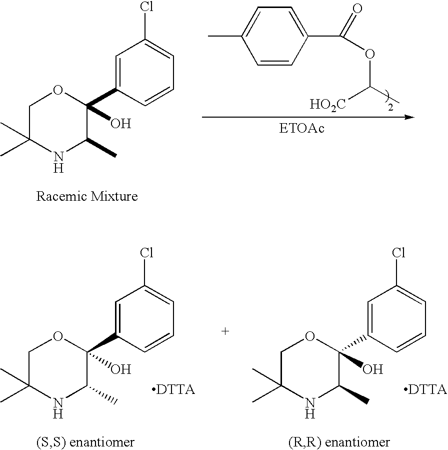

### Example 2

(2R*,3R*) racemate (a 50/50 mixture of the (2R,3R) and (2S,3S) enantiomers, 0.5 g) was dissolved in 5 mL of the solvent described in Table 1, below, then added to a stirred solution of L-DTTA (1.13 grams, 1.5 equiv) in 3 mL of the same solvent in a heating bath at 80° C. The mixture was stirred and heated for 18 hours, then cooled. The product was filtered off, washed with fresh solvent and dried to give product having the enantiomer ratio described in the following Table 1.

TABLE 1
Resolution of the (2R*, 3R*) racemate in various solvents

| | | Isomer Ratio 2S, 3S: |
|---|---|---|
| Example | Solvent | 2R, 3R |
| 2A | Methyl Acetate | 99.6:0.4 |
| 2B | Isopropyl Acetate | 99.6:0.4 |
| 2C | Propyl Acetate | 99.6:0.4 |
| 2D | Isobutyl Acetate | 98.6:1.4 |
| 2E | Butyl Acetate | 99.0:1.0 |
| 2F | Ethyl Acetate | 99.7:0.3 |
| 2G | 2,4-Dimethyl-3-Pentanone | 99.6:0.4 |
| 2H | 3-Methyl-2-Butanone | 99.8:0.2 |
| 2I | 2-Butanone | 99.9:0.1 |
| 2J | 4-Methyl-2-Pentanone | 99.7:0.3 |
| 2K | Acetonitrile | 99.8:0.2 |
| 2L | Propionitrile | 99.9:0.1 |

The yields of the required (2S,3S) enantiomer from these Examples is given in the following Table 2.

TABLE 2

| Example | Yield (%) |
|---|---|
| 2A | 54 |
| 2B | 92 |
| 2C | 83 |
| 2D | 97 |
| 2E | 89 |
| 2F | 90 |
| 2G | 62 |
| 2H | 71 |
| 2I | 55 |
| 2J | 73 |
| 2K | 63 |
| 2L | 63 |

The quoted yield for Example 21 was achieved by using a higher concentration of racemate (reducing the solvent volume to approximately half of that indicated above), due in part to the fact that the (2S,3S)-enantiomer is more soluble in the particular solvent concerned (2-butanone) compared to the other solvents referred to, and also due to a degree of degradation at the lower concentration. Similarly, the recovery of the (2S,3S)-enantiomer from the other solvents giving moderate yields (Examples 2A, 2G, 2K, 2L) would be expected to be improved if the experiment was performed using higher concentrations (lower relative solvent volumes). In addition, the yield for Example 2A would be expected to be improved if the experiment was performed using a longer time for reflux given that the boiling point of the solvent is relatively low.

### Example 3

A sample of the (2R,3R) enantiomer (0.5 g) was dissolved in ethyl acetate (5 ml) then added to a stirred boiling solution of L-DTTA (1.13 g, 1.5 equiv) in ethyl acetate (3 ml). The mixture was heated at reflux for 18 hours then cooled. The product was filtered off, washed with ethyl acetate and dried to give a 70% yield of the L-DTTA salt of the (2S,3S) enantiomer.

### Comparative Examples

A procedure analogous to that of Example 2 was followed using other solvents to give a product having the enantiomer ratio and overall yield as described in the following Table 3.

TABLE 3

| Example | Solvent | Isomer Ratio 2S, 3S:2R, 3R | Yield (%) |
|---|---|---|---|
| C1 | Diethylene Glycol | 99.8:0.2 | 19 |
| C2 | tert-Butanol | 50:50 | 21 |

All cited patents, publications, co-pending applications, and provisional applications referred to in this application are herein incorporated by reference.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A process for preparing a salt of (+)-(2S,3S)-2-(3-chlorophenyl)-3,5,5-trimethyl-2-morpholinol that comprises:
mixing i) a sample comprising (−)-(2R,3R)-2-(3-chlorophenyl)-3,5,5-trimethyl-2-morpholinol ((2R,3R) enantiomer), ii) at least one solvent having a boiling point of at least 50° C. and iii) 1.1 equivalent or higher of L-DTTA in any order, heating the mixture to at least 50° C. for at least 1 hour to form crystals comprising an L-DTTA salt of (+)-(2S,3S)-2-(3-chlorophenyl)-3,5,5-trimethyl-2-morpholinol ((2S,3S) enantiomer), and isolating the crystals, wherein the yield of the L-DTTA salt of the (2S,3S) enantiomer is greater than 50% based on said sample.

2. The process according to claim 1, wherein the solvent preferably dissolves the L-DTTA salt of the (2R,3R) enantiomer over the L-DTTA salt of the (2S,3S) enantiomer.

3. The process according to claim 1, wherein the solvent is at least one selected from alkyl acetate, dialkyl ketone, and nitrile.

4. The process according to claim 3 wherein the solvent is ethyl acetate.

5. The process according to claim claim 1, wherein the amount of L-DTTA is 1.2-2.0 equivalents.

6. The process according to claim 1, wherein the mixture of the sample comprising the (2R,3R) enantiomer, solvent and L-DTTA is heated to reflux.

7. The process according to claim 1, wherein the mixture is heated for at least 5 hours.

8. The process according to claim 1, wherein the crystals are essentially enantiomerically pure with respect to the (2S,3S) enantiomer.

9. The process according to claim 1, which is a continuous process.

10. The process according to claim 1, wherein the sample comprising the (2R,3R) enantiomer is a racemic mixture of the (2R,3R) enantiomer and the (2S,3S) enantiomer.

11. The process according to claim 1, wherein the sample comprising the (2R,3R) enantiomer is a non-racemic mixture of the (2R,3R) enantiomer and the (2S,3S) enantiomer.

12. The process according to claim 1, wherein said sample comprising the (2R,3R) enantiomer contains at least 50 wt % of the (2R,3R) enantiomer based on the weight of said sample.

13. The process according to claim 1, wherein the sample comprising the (2R,3R) enantiomer is essentially enantiomerically pure (2R,3R) enantiomer.

14. The process according to claim 1, wherein said sample comprising the (2R,3R) enantiomer is formed in a step comprising reacting 2-bromo-3′-chloropropiophenone with 2-amino-2-methylpropanol.

15. The process according to claim 1, further comprising a step of converting the L-DTTA salt of the (2S,3S) enantiomer to another salt which is pharmaceutically acceptable.

16. The process according to claim 15, wherein the other salt is a hydrochloride salt.
